(19) United States
(12) Patent Application Publication
(10) Pub. No.: US 2023/0052390 A1
(43) Pub. Date: Feb. 16, 2023

(54) BIOLOGICAL INFORMATION ACQUISITION DEVICE AND BIOLOGICAL INFORMATION ACQUISITION METHOD

(71) Applicant: OMRON HEALTHCARE Co., Ltd., Kyoto (JP)

(72) Inventors: Daisuke ISHIHARA, Kyoto (JP); Miki OKUGAWA, Kyoto (JP); Mizuki SHIMOGAMA, Kyoto (JP)

(21) Appl. No.: 17/932,170

(22) Filed: Sep. 14, 2022

Related U.S. Application Data
(63) Continuation of application No. PCT/JP2021/008559, filed on Mar. 4, 2021.

(30) Foreign Application Priority Data
Mar. 19, 2020 (JP) 2020-050161

(51) Int. Cl. G16H 10/20

(52) U.S. Cl. CPC G16H 10/20

(57) ABSTRACT

A state of a cognitive function can be determined by using biological information measured as part of daily healthcare management. A biological information acquisition device includes a cognitive function test unit for testing a cognitive function, a biological information acquisition unit for performing biological information acquisition processing for acquiring biological information of a user, and a cognitive function determination unit for determining a state of the cognitive function, wherein the cognitive function determination unit determines the state of the cognitive function based on a result of the cognitive function test and the biological information acquired from the user.

### CROSS-REFERENCE TO RELATED APPLICATIONS

This application is the U.S. national stage application filed pursuant to 35 U.S.C. 365(c) and 120 as a continuation of International Patent Application No. PCT/JP2021/008559, filed Mar. 4, 2021, which application claims priority to Japanese Patent Application No 2020-050161, filed Mar. 19, 2020, which applications are incorporated herein by reference in their entireties.

### TECHNICAL FIELD

The present invention relates to a biological information acquisition device and a biological information acquisition method.

### BACKGROUND ART

In recent years, approach to dementia has been a challenge. Dementia is considered to refer to a condition in which an intellectual function temporarily normally developed is degraded persistently and a plurality of intellectual functions including a memory function are impaired, and thus social life is interfered with. Impairment of the intellectual functions in dementia varies from a mild stage to a severe stage and is considered to advance in a step-wise fashion. Dementia advanced to a moderate stage interferes with daily life, but in a mild dementia state, lifestyle improvement and an anti-dementia drug may be effective, and thus early detection and early treatment are important.

As a cognitive function test for early detection of dementia, various tests have been proposed such as MMSE, Hasegawa Dementia Scale-Revised (HDS-R), WMS-R, ADAS-J, Cog, MoCA-J, and FCSRT. In a case of performing a diagnosis of dementia in a medical institution, a plurality of the cognitive function tests of these are performed in combination, and neurological consultation, MRI, and the like are also performed. Since it takes time and effort to diagnose dementia as described above, there is a high hurdle for a person who receives a dementia test, and this is one of the reasons that prevents early detection.

Patent Document 1 proposes a dementia diagnosis assistance system that provides a simplified diagnostic test for dementia in a short time during a waiting time in a medical institution.

### CITATION LIST—PATENT LITERATURE

Patent Document 1: JP 2017-217051 A

### SUMMARY OF INVENTION

### Technical Problem

In some cases, biological information measured in daily healthcare management may include an element considered to be changed prior to or accompanied by an onset of dementia or a change in a state of a cognitive function. In view of the circumstances described above, an object of the present invention is to provide a biological information acquisition device capable of determining the change in the state of the cognitive function using biological information measured as a part of daily healthcare management.

### Solution to Problem

In order to solve the problem described above, a biological information acquisition device according to the present invention is a biological information acquisition device configured to perform a cognitive function test, the biological information acquisition device including a cognitive function test unit configured to test a cognitive function, a biological information acquisition unit configured to perform biological information acquisition processing to acquire biological information of the user, and a cognitive function determination unit configured to determine a state of the cognitive function, wherein the cognitive function determination unit determines the state of the cognitive function based on a result of the cognitive function test and the biological information acquired from a user.

In this manner, it is possible to determine the cognitive function in consideration of an element that is included in biological information measured in daily healthcare management and that is considered to be changed prior to or accompanied by the onset of dementia or a change in the state of the cognitive function. As used herein, the term “changed prior to or accompanied” is intended to include the following. That is, of the main cause of change is not limited to “onset of dementia” and “change in a state of a cognitive function” (meaning there is a so-called causal relationship). It can also include cases where each of them changes due to another factor, for example, spurious correlation. In addition, it is not necessarily required to be temporally close to each other, and for example, information related to a past event of the subject such as “a state of chronic exercise deficiency” or “presence or absence of a past surgical history” can also be determined as a risk.

Further, the biological information includes, but is not limited to, blood pressure information such as systolic blood pressure, diastolic blood pressure, and pulse, body composition such as body weight and body fat percentage, exercise amount such as a number of steps and calorie consumption, heart rate, electrocardiographic data, respiratory rate, body temperature, and the like.

Additionally, as a cognitive function test, an appropriate cognitive function test such as a test dedicated to memorization can be employed with reference to various tests such as MMSE, Hasegawa Dementia Scale-Revised (HDS-R), WMS-R, ADAS-J, Cog, MoCA-J, and FCSRT, which are well known.

Further, in the present invention, an attribute information acquisition unit configured to acquire attribute information related to an attribute of the user may be provided, wherein the cognitive function determination unit further determines the state of the cognitive function of the user based on the attribute information related to the attribute.

In this manner, it is possible to determine the cognitive function reflecting the element that is included in the attribute information related to the physical characteristics or the daily lifestyle of the user and that is considered to be changed prior to or accompanied by the onset of dementia or the change in the state of the cognitive function.

Here, the attribute information includes, but is not limited to, age, gender, presence or absence of smoking, presence or absence of drinking, obesity level, exercise habits, and the like.

In addition, in the present invention, the biometric the biological information acquisition unit may acquire a plurality of types of biological information related to the user, and the cognitive function determination unit may further determine the state of the cognitive function based on the plurality of types of biological information acquired by the biological information acquisition unit.

In this manner, it is possible to determine the cognitive function reflecting the element related to a plurality of types of biological information that is considered to be changed prior to or accompanied by the onset of dementia or a change in the state of the cognitive function.

Further, in the present invention, a communication unit connected to another biological information measurement instrument may be further provided, wherein the biological information acquisition unit may acquire at least one or more pieces of biological information measured by the other biological information measurement instrument connected through the communication unit, and the attribute information acquisition unit may acquire the attribute information related to the attribute of the user via the other biological information measurement instrument connected through the communication unit.

In this manner, the plurality of types of attribute information and biological information related to the user can be acquired via the other biological information device connected to the communication unit. The determination accuracy of the cognitive function can be enhanced on the basis of the plurality of types of biological information and attribute information related to the physical characteristics and lifestyle habits of the user, which are considered to be changed prior to or accompanied by the onset of dementia or a change in the state of the cognitive function.

In addition, in the present invention, the cognitive function determination unit may use a determination threshold for determining the cognitive function and correct the determination threshold based on the result of the cognitive function test and the biological information acquired from the user. Further, the cognitive function determination unit may further correct the determination threshold based on the attribute information related to the attribute of the user. Further, the cognitive function determination unit may further correct the determination threshold based on the plurality of types of biological information acquired by the biological information acquisition unit. Even in such a form, it is possible to determine the cognitive function reflecting the element that is considered to be changed prior to or accompanied by the onset of dementia or a change in the state of the cognitive function.

Furthermore, the present invention can be grasped as a biological information acquisition method capable of performing a cognitive function test, the biological information acquisition method including testing the cognitive function of a user, acquiring biological information of the user, and determining a state of the cognitive function of the user based on a result of the cognitive function test and the biological information acquired from the user.

In this manner, it is possible to determine the cognitive function corrected in consideration of the element that is included in biological information measured in daily healthcare management and that is considered to be changed prior to or accompanied by the onset of dementia or a change in the state of the cognitive function.

In this regard, the biological information includes, but is not limited to, blood pressure information such as systolic blood pressure, diastolic blood pressure, and pulse, body composition such as body weight and body fat percentage, exercise amount such as the number of steps and calorie consumption, heart rate, electrocardiographic data, respiratory rate, body temperature, and the like.

Additionally, as a cognitive function test, an appropriate cognitive function test such as a test dedicated to memorization can be employed with reference to various tests such as MMSE, Hasegawa Dementia Scale-Revised (HDS-R), WMS-R, ADAS-J, Cog, MoCA-J, and FCSRT, which are well known.

### Advantageous Effects of Invention

According to the present invention, it is possible to provide a biological information acquisition device capable of determining a state of a cognitive function using biological information measured as a part of daily healthcare management.

### BRIEF DESCRIPTION OF DRAWINGS

Various embodiments are disclosed, by way of example only, with reference to the accompanying schematic drawings in which corresponding reference symbols indicate corresponding parts, in which:

FIG. 1 is a block diagram illustrating an overview of a configuration example of a biological information measurement system according to a first embodiment;

FIG. 2 is a flowchart illustrating a processing procedure for a blood pressure information measurement method of a biological information measurement system according to the first embodiment;

FIG. 3 is a flowchart illustrating a processing procedure for provision of test information in the biological information measurement system according to the first embodiment;

FIG. 4 is a diagram illustrating a display example of a touch panel display of the biological information measurement system according to the first embodiment;

FIG. 5 is a diagram illustrating transition of words displayed in provision of test information in the biological information measurement system according to the first embodiment;

FIG. 6 is a flowchart illustrating a procedure for blood pressure information measurement processing using a blood pressure monitor of the biological information measurement system according to the first embodiment;

FIG. 7 is a diagram illustrating a display example of a touch panel display in the blood pressure information measurement processing using the blood pressure monitor of the biological information measurement system according to the first embodiment;

FIG. 8 is a flowchart illustrating a processing procedure for answer check in the biological information measurement system according to the first embodiment;

FIG. 9 is a flowchart illustrating a processing procedure of a determination processing on a result of a test performed by the biological information measurement system according to the first embodiment;

FIGS. 10A and 10B are diagrams illustrating an example of a determination correction table according to the first embodiment;

FIG. 11 is a diagram illustrating an example of a determination correction table according to the first embodiment;

FIG. 12 is a diagram illustrating transition of words displayed in provision of test information in the biological information measurement system according to a second embodiment;

FIG. 13 is a flowchart illustrating a processing procedure for answer check in the biological information measurement system according to the second embodiment;

FIG. 14 is a diagram illustrating transition of words provided in answer check processing of the biological information measurement system according to the second embodiment;

FIG. 15 is a flowchart illustrating a processing procedure for provision of test information in a biological information measurement system according to a third embodiment;

FIG. 16 is a diagram illustrating a display example of a touch panel display in the test information provision processing according to the third embodiment;

FIG. 17 is a diagram illustrating transition of illustrations provided in the test information provision processing according to the third embodiment;

FIG. 18 is a flowchart illustrating a processing procedure for answer check in the biological information measurement system according to the third embodiment;

FIG. 19 is a flowchart illustrating a processing procedure for provision of test information in a biological information measurement system according to a fourth embodiment; and,

FIG. 20 is a flowchart illustrating a processing procedure for reception of transferred biological information/attribute information in the biological information measurement system according to the fourth embodiment.

### DESCRIPTION OF EMBODIMENTS

Embodiments of the present invention will be specifically described below with reference to the drawings.

### First Embodiment

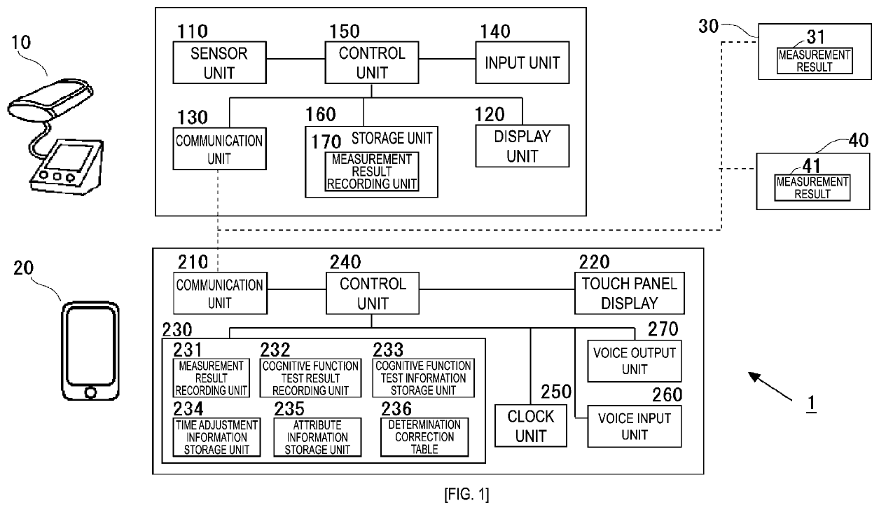

First, an example of the embodiment of the present invention will be described based on FIG. 1 to FIG. 11. It should be noted that the dimension, material, shape, relative arrangement and the like of the components described in the present embodiment are not intended to limit the scope of this invention to them alone, unless otherwise stated.

### System Configuration

FIG. 1 is a schematic diagram illustrating a configuration example of a biological information measurement system 1 used as a biological information acquisition device according to the present embodiment. As illustrated in FIG. 1, the biological information measurement system 1 includes a blood pressure monitor 10 used as a biological information measurement instrument, and a smartphone 20 used as a terminal. The blood pressure monitor 10 and the smartphone 20 are configured to be communicable with each other. A communication method is not particularly limited, and the communication method employed may include a wireless communication method such as Bluetooth (trade name), infrared communication, or information transmission using ultrasonic waves, or a wired communication method with connection via a cable, a connector, or the like.

In FIG. 1, the biological information measurement instruments 30, 40 are communicably connected to the smartphone 20. Here, a body weight meter measuring the body weight of the user is exemplified as the biological information measurement instrument 30. However, the biological information measurement instrument 30 may measure a body composition such as a body fat percentage in addition to a body weight, and may also calculate a Body Mass Index (BMI) value which is a rough indication of the obesity level based on attribute information such as age, gender, and height input from the user. In the present embodiment, the attribute information includes general information indicating the lifestyle of the user. In addition, an exercise meter that measures an exercise amount such as a number of steps or consumed calories is exemplified as the biological information measurement instrument 40. In the biological information measurement instrument 40, attribute information such as age, gender, body weight, height, and presence or absence of exercise habits of the user may be input in order to calculate an exercise amount such as calorie consumption. In the biological information measurement instrument 30, for example, a measurement result 31 of the measured body weight, body fat percentage, BMI, or the like is recorded in the memory included in the own device together with the time information at which the measurement is performed and the attribute information. In the same manner as in the biological information measurement instrument 40, the measured exercise amount such as the number of steps and consumed calories is recorded as a measurement result 41 in the memory of the own device together with the time information at which the measurement is performed and the attribute information. Note that in each biological information measurement instrument, the recorded measurement result is held as a measurement history for a certain period of time.

Hereinafter, the biological information measurement system according to the present embodiment will be described using the blood pressure monitor 10 as a representative example of the biological information measurement instrument.

### Blood Pressure Monitor

The blood pressure monitor 10 of the present embodiment is a measurement apparatus that measures a blood pressure of a user using a so-called oscillometric method and, as illustrated in FIG. 1, includes a sensor unit 110, a display unit 120, a communication unit 130, an input unit 140, a control unit 150, and a storage unit 160.

The sensor unit 110 includes a pressure sensor disposed in a cuff portion of the blood pressure monitor 10, and detects a pulse wave from a blood vessel of the user under a proper cuff pressure. The blood pressure monitor 10 in the present embodiment can measure pulses in addition to the systolic blood pressure and diastolic blood pressure on the basis of pulse waves detected by the sensor unit. In the following description, the values of the systolic blood pressure, diastolic blood pressure, and pulse are collectively referred to as blood pressure information. In the present embodiment, the blood pressure information corresponds to the biological information.

The display unit 120, which is formed of a liquid crystal display or the like for example, displays calculated blood pressure information.

The communication unit 130 is a communication interface that manages transmission and reception of signals to and from the smartphone 20. In a case of using the wireless communication method, the communication unit 130 is a communication antenna that transmits and receives radio waves including radio waves for short-range wireless communication. However, the communication unit 130 is not limited to the communication antenna, and a desired known technology can be employed. Additionally, the communication unit 130 may use the wired communication method, and a desired known technology can be employed. The communication unit 130 transmits information including the inherent identification number (such as the serial number) of each blood pressure monitor, and the measured blood pressure information.

The input unit 140 is an input unit such as a button or a touch panel display that receives input from the user, and receives various operations from the user, such as power-on and -off, initiation of measurement, selection of an item, and input of attribute information.

The control unit 150 is a unit that manages the control of the blood pressure monitor 10, and includes a central processing unit (CPU) and the like, for example. In response to reception of an instruction to initiate measurement, from the user via the input unit 140, the control unit 150 pressurizes the cuff and, under an appropriate cuff pressure, calculates the blood pressure information based on the pulse wave detected by the sensor unit 110. Then, the calculated value is displayed on the display unit 120. In addition, each component of the blood pressure monitor 10 is controlled to execute processing corresponding to the operation of the user via the input unit 140.

A storage unit 160 includes, for example, a long-term storage medium such as a flash memory, in addition to a main storage device such as a random access memory (RAM), and stores various types of information such as application programs and the blood pressure information.

A measurement result recording unit 170 is a non-volatile memory included in the storage unit 160, and records information such as blood pressure information measured by the sensor unit 110.

### Smartphone

As illustrated in FIG. 1, the smartphone 20 includes a communication unit 210, a touch panel display 220, a storage unit 230, a control unit 240, a clock unit 250, a voice input unit 260, and a voice output unit 270.

The communication unit 210 is a communication interface that manages transmission and reception of signals to and from the blood pressure monitor 10. In a case of using the wireless communication method, the communication unit 210 is a communication antenna that transmits and receives radio waves including radio waves for short-range wireless communication. In addition to receiving measurement information transmitted from the blood pressure monitor 10, the communication unit 210 also transmits and receives radio waves to and from other electronic apparatuses or base stations. The communication unit 210 may use the wired communication method, and a desired known technology can be employed.

The touch panel display 220 is used both as a display unit and as an input unit. The touch panel display 220 displays various pieces of information to the user, and receives various inputs from the user.

The storage unit 230 includes, for example, a long-term storage medium such as a flash memory, in addition to a main storage device such as a RAM, and stores various types of information such as application programs and the biological information.

A measurement result recording unit 231 is a non-volatile memory included in the storage unit 230, and information recorded in the measurement result recording unit 231 includes blood pressure information received from the blood pressure monitor 10 and information such as measurement date and time at which the blood pressure information is measured.

A cognitive function test result recording unit 232 is a non-volatile memory included in the storage unit 230, and the results of the cognitive function test described below are recorded in the cognitive function test result recording unit 232. The results of the cognitive function test include, but are not limited to, information of the dates and times at which the cognitive function tests are performed, the contents of the test, and evaluation information such as the correct answer rate.

A cognitive function test information storage unit 233 is a non-volatile memory included in the storage unit 230, and stores data such as words and illustrations used in the cognitive function test and programs for conducting the cognitive function test. Additionally, in accordance with the contents of the cognitive function test, information is also stored that is related to the amount of time that needs to elapse, which is required after provision of test information until check of answers from the user to the test information.

A time adjustment information storage unit 234 is a non-volatile memory included in the storage unit 230. The time adjustment information storage unit 234 stores time adjustment information used to adjust blood pressure measurement time and the amount of time after the execution of the cognitive function test until the check of the answers from the user. Specifically, the time adjustment information is information provided to the user through the touch panel display 220 and the voice output unit 270, and in order to provide information in accordance with a necessary adjustment time, various types of information, such as visual effects for display of measurement values, music, moving images, programs for conversations with characters are stored.

An attribute information storage unit 235 is a non-volatile memory included in the storage unit 230. The attribute information storage unit 235 stores, for example, attribute information such as age, smoking status, educational history, and exercise habits. However, the attribute information includes general information indicating the “lifestyle” of the user. Further, the “educational history” may include “medical history”, “history related to occupation”, and “information related to life history” of the user.

Here, a part of the attribute information may be acquired via a network to which the smartphone 20 can be connected. For example, attribute information such as the obesity level, the measured amount of exercise, and the frequency of exercise can be acquired from the biological information measurement instruments 30, 40 connected through the communication unit 210.

A determination correction table 236 is a non-volatile memory included in the storage unit 230. The determination correction table 236 stores correction information such as a correction factor for correcting a threshold for determining a state of a cognitive function. In the present embodiment, a threshold for determining the state of the cognitive function is determined based on the determination correction table 236, the measurement result of the biological information, and the attribute information.

The threshold changes according to the measurement result of the biological information and the attribute information. Then, the state of the user's cognitive function is determined based on the result of the cognitive function test and the determined threshold.

The control unit 240 is a means that manages the control of the smartphone 20, which, for example, includes a CPU and the like and executes various programs stored in the storage unit 230 to serve functions of them.

The clock unit 250 has a timing function, and holds the current date/time information.

The voice input unit 260 includes a microphone that converts voice into an electrical signal, and receives input of voice information.

The voice output unit 270 includes a speaker that converts an electrical signal into voice, and outputs voice information.

### Biological Information Acquisition Method

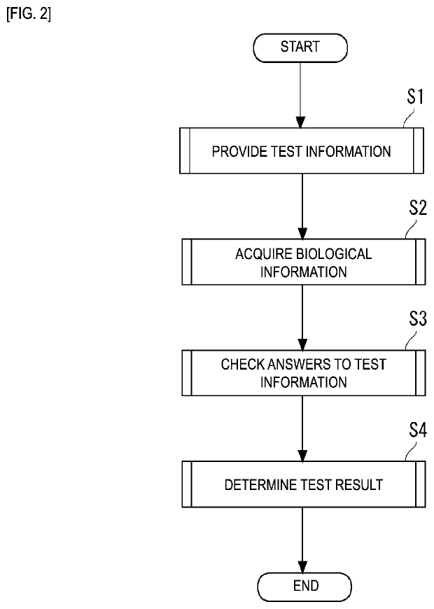

FIG. 2 is a flowchart illustrating a processing procedure for a biological information acquisition method including cognitive function test processing, biological information acquisition processing, answer check processing, and test result determination processing. In this regard, blood pressure information used as biological information is acquired by measurement.

First, information for the cognitive function test is provided to the user via the smartphone 20 (step S1).

Then, blood pressure information, used as biological information, is measured by the blood pressure monitor 10 (step S2).

Then, answers to information for the cognitive function test are received from the user and checked, via the smartphone 20 (step S3).

Then, the state of the cognitive function is determined based on the acquired biological information and the result of the cognitive function test (step S4).

### Cognitive Function Test Information Provision Processing

As a cognitive function test, various tests have been proposed such as MMSE, Hasegawa Dementia Scale-Revised (HDS-R), WMS-R, ADAS-J, Cog, MoCA-J, and FCSRT. In the present embodiment, in order to effectively utilize the amount of time during which the blood pressure information is measured using the blood pressure monitor 10, a test dedicated to memorization is conducted, but the present invention is not limited to such a test. When the cognitive function test is conducted, in a case where a predetermined amount of time is required to elapse before the user answers or a predetermined interference problem is required, the user can be prompted to measure the blood pressure information or to perform tasks including the measurement as an alternative to the elapse of the predetermined amount of time or the interference problem. This allows the cognitive function test to be readily conducted in conjunction with the measurement of the blood pressure information that is routine healthcare management. This in turn enables easing of uncomfortable feeling in taking the cognitive function test and also enables a reduction in time and effort required to take the cognitive function test, allowing early detection of dementia to be expected.

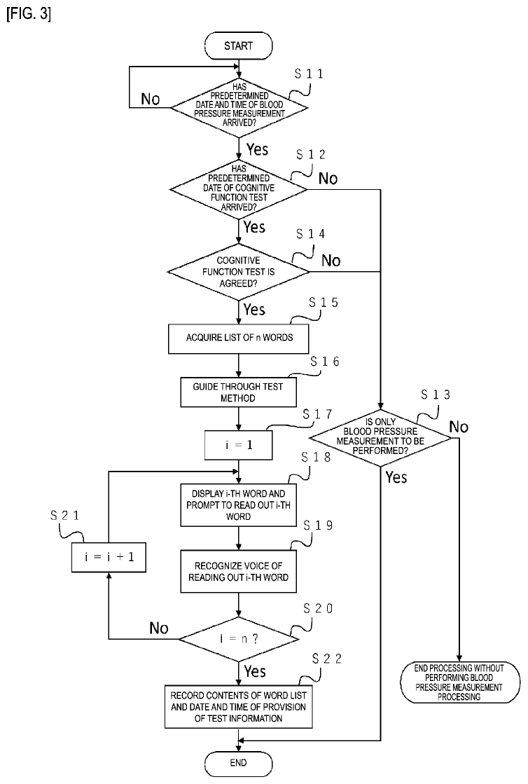

FIG. 3 is a flowchart illustrating a processing procedure for provision of cognitive function test information. The cognitive function test of the present embodiment is referred to as word recalling, in which the user is prompted to memorize a plurality of displayed words and to orally speak the previously displayed words after a predetermined amount of time or after a predetermined interference task. The cognitive function test information provision processing described below, including other examples, is implemented by the control unit 240 of the smartphone 20 by executing a program stored in the storage unit 230. In other words, a test information provision unit is configured by including the touch panel display 220, the storage unit 230, the clock unit 250, the voice input unit 260, and the voice output unit 270, and the control unit 240 cooperating with the units 220, 230, 250, 260, and 270. Additionally, the cognitive function test function is implemented by executing the cognitive function test information provision processing and the answer check processing described below.

When the date and time preset by the user as the date and time of blood pressure information measurement arrives (step S11), whether the date preset as the date of the cognitive function test has arrived is determined (step S12).

In a case where in step S12, the date of the cognitive function test is determined not to have arrived, the user is prompted via the touch panel display 220 to determine whether to perform only blood pressure measurement (step S13). In step S13, in a case where an input indicating that only blood pressure measurement is to be performed is received from the user via the touch panel display 220 or via the voice input unit 260, the cognitive function test information provision processing is ended and blood pressure measurement processing described below is performed. In this case, when the blood pressure measurement processing ends, the processing is ended without execution of the answer check processing. In step S13, in a case where an input indicating that only blood pressure measurement is not to be performed is received from the user via the touch panel display 220 or via the voice input unit 260, the processing is ended without the blood pressure measurement processing being executed.

In a case where it is determined in step S12 that the date of the cognitive function test has arrived, then a message “Would you like to take the cognitive level test?” is displayed on the touch panel display 220 of the smartphone 20, or the voice output unit 270 outputs the voice of the corresponding message and asks the user for agreement (step S14).

At this time, a “yes” and a “no” buttons are displayed on the touch panel display 220 of the smartphone 20, and which button the user has touched is detected, and whether the user has agreed to take the test is determined. Alternatively, determination may be performed by receiving an oral answer of the user from the voice input unit 260 of the smartphone 20.

In a case where the user does not agree, then the processing proceeds to step S13.

In a case where the user agrees, a list of n words stored in the cognitive function test information storage unit 233 of the storage unit 230 is acquired (step S15). The cognitive function test information storage unit 233 may store a word list, or may be configured to extract n words from a group of words to constitute a word list.

Then, a cognitive function test method to be performed is displayed on the touch panel display 220 of the smartphone 20 to guide through the method (step S16). Here, a message for guiding a method of the cognitive function test such as “From now, n words will be displayed one by one. Please read out and memorize the displayed words” is displayed. The message may also be output by the voice output unit 270 of the smartphone 20.

Next, i=1 is set (step S17).

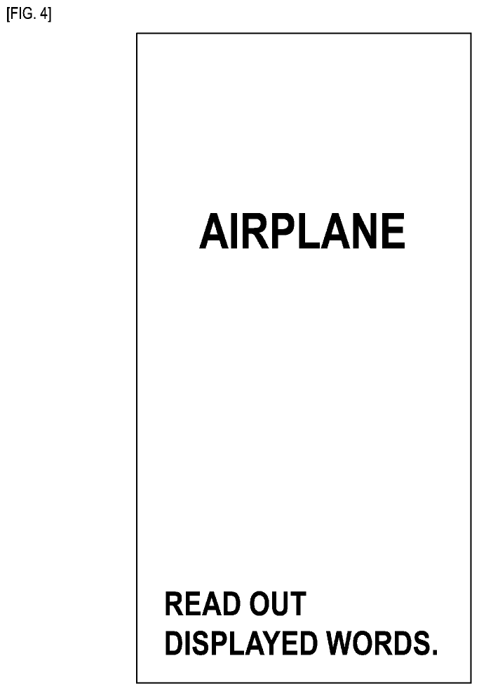

As illustrated in FIG. 4, the first word of the word list acquired in step S15 is displayed on the touch panel display 220, and a message prompting the user to read out the displayed word is similarly displayed (step S18). In this case, the word “airplane” is displayed on the touch panel display 220. The message prompting to read out the displayed word may be output via the voice output unit 270.

From the voice acquired via the voice input unit 260 of the smartphone 20, the voice of the user reading out the i-th word is recognized (step S19). In a case where the voice of the user reading out the i-th word fails to be recognized within a predetermined amount of time, then a message prompting the user to read out the displayed word may be displayed on the touch panel display 220 or output via the voice output unit 270. Additionally, in a case where the voice of the user reading out the i-th word fails to be recognized within the predetermined amount of time, the processing may proceed to step S20.

When the voice of the user reading out the i-th word is recognized, whether i=n is determined (step S20).

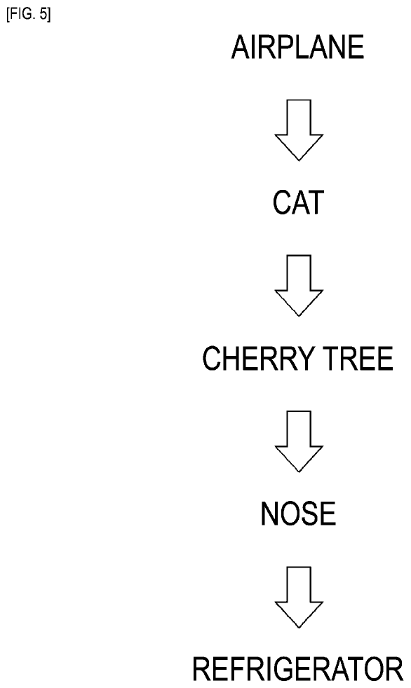

In step S20, if i=n is not satisfied, i=i+1 is set (step S21), the process returns to step S18, and the processing after step S18 is repeated for the next word in the word list acquired in step S15. In this manner, as illustrated in FIG. 5, the different words are sequentially displayed on the touch panel display 220 of the smartphone 20. In this case, the word list is constituted by five words of an airplane, a cat, a cherry tree, a nose, and a refrigerator. The words displayed at this time all belong to different categories, such as a vehicle, an animal, a flower, a part of a body, a home electrical appliance, and the like. The number n of words displayed is not limited to 5, and may be set as appropriate. In a case where similar tests are repeated a plurality of times, the number of displayed words increases each time, for example, five words on the first test and seven words on the second test.

If i=n in step S20, the contents of the word list acquired in step S15 and the date and time when the test information was provided are stored in the cognitive function test result recording unit 232 of the storage unit 230 (step S22). In this case, the date and time of provision of the test information is, for example, the date and time of recognition of the voice of reading out the nth word.

Then, the provision processing for the cognitive function test information is ended.

When the provision processing for the cognitive function test information ends as described above, the processing proceeds to the blood pressure information measurement processing (step S2), as illustrated in FIG. 2.

### Blood Pressure Information Measurement Processing

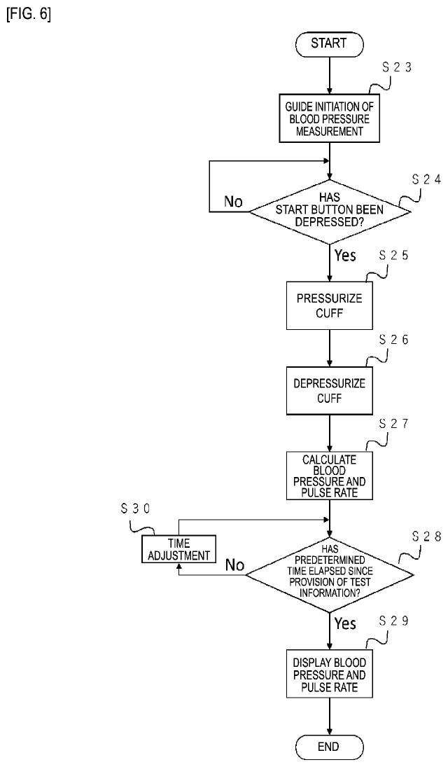

FIG. 6 is a flowchart illustrating a processing procedure for measurement of blood pressure information. The blood pressure information measurement processing described below, including other examples, is implemented by the control unit 240 of the smartphone 20 by executing a program stored in the storage unit 230 and cooperating with the blood pressure monitor 10. At this time, in the blood pressure monitor 10, the control unit 150 similarly executes a program stored in the storage unit 160. The biological information measuring unit used as a biological information acquisition unit includes the touch panel display 220, the storage unit 230, the clock unit 250, the voice input unit 260 and the voice output unit 270, the communication unit 210, and the control unit 240 that are belonging to the smartphone 20, in which the control unit 240 cooperates with the units 220, 230, 250, 260, 270, and 210. The biological information measuring unit further includes at least the sensor unit 110, the storage unit 160, the communication unit 130, and the control unit 150 that are belonging to the blood pressure monitor 10, in which the control unit 150 cooperates with the units 110, 160, and 130.

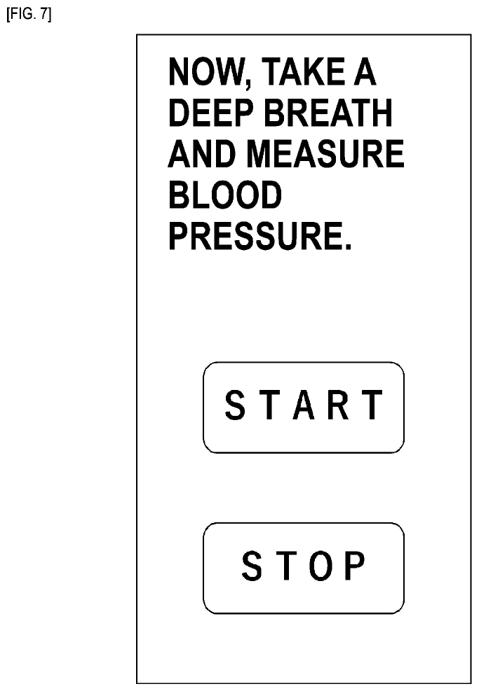

First, the user is guided to the initiation of the blood pressure measurement (step S23). At this time, as illustrated in FIG. 7, a “START” button and a “STOP” button are displayed on the touch panel display 220 of the smartphone 20, and a message prompting blood pressure information measurement, as “Now, take a deep breath and measure the blood pressure.” is displayed on the above touch panel display 220 or output from the voice output unit 270. Here, a message instructing the user to wrap the cuff around the arm may be displayed on the touch panel display 220 or output from the voice output unit 270. At this time, predetermined initialization of the blood pressure monitor 10 is performed.

The smartphone 20 waits until the user wraps the cuff around the arm and depresses the “START” button displayed on the touch panel display 220 of the smartphone 20 or the user inputs the voice “start” via the voice input unit 260 (step S24).

When depression of the “START” button or the voice “start” is detected, the cuff pressure is increased (step S25). When the cuff is pressurized to a predetermined pressure, the cuff pressure is gradually reduced (step S26).

Then, a known method is used to calculate the systolic blood pressure, the diastolic blood pressure, and the pulse rate (step S27).

Then, it is determined whether a predetermined amount of time has elapsed since the date and time of provision of the cognitive function test information stored in step S22 (step S28). The predetermined amount of time is the amount of time to be unoccupied from provision of the cognitive function test information, such as display of words, until the check of answers such as recalling of words by the user, in accordance with the contents of the cognitive function test performed in step S1. Such a predetermined time is stored in the cognitive function test information storage unit 233 of the storage unit 230.

In step S28, in a case where the determination is made that the predetermined amount of time has elapsed since the date and time of provision of cognitive function test information, the systolic blood pressure, diastolic blood pressure, and pulse rate calculated in step S27 are displayed on the touch panel display 220 of the smartphone 20 (step S29), and the processing is ended. In step S29, the calculated systolic blood pressure, diastolic blood pressure, and pulse rate may be output via the voice output unit 270.

In step S28, in a case where the determination is made that the predetermined amount of time has not elapsed since the date and time of provision of cognitive function test information, the processing proceeds to step S30. In step S30, time adjustment processing is performed to adjust the time elapsing from the date and time of provision of cognitive function test information. In this regard, as an example of the time adjustment processing, time adjustment information stored in the time adjustment information storage unit 234 of the storage unit 230 is provided to the user via the touch panel display 220 and the voice output unit 270. Here, the content of the time adjustment information is selected according to the difference between the time to be reserved from the provision of the cognitive function test information to the check of the answer and the elapsed time up to the present. The time of the blood pressure information measurement processing is determined including the time to provide the selected time adjustment information. The time adjustment information is, for example, music, moving images, games, conversations with characters, and the like, and these files and programs are read from the time adjustment information storage unit 234 and reproduced or executed to provide the time adjustment information to the user. These pieces of time adjustment information are provided, and the elapse of a predetermined amount of time is waited for since the date and time of provision of the cognitive function test information. In accordance with the difference described above, a plurality of types of information may be provided, or the provision may be stopped partway through one piece of information. The time adjustment information is not limited to the time adjustment information stored in the time adjustment information storage unit 234, and may be provided to the user by a method such as streaming from an external server via a network.

In this case, the time adjustment information is provided before display of the blood pressure information on the touch panel display 220 (step S29). However, by displaying, on a graph, a history including previous blood pressure information recorded in the measurement result recording unit 231 of the storage unit 230, along with display of the blood pressure information such as the systolic blood pressure acquired by the current measurement, or applying visual or auditory effects to the display of the current blood pressure information, or the like, the time adjustment information may be provided including information related to the current blood pressure information. Additionally, for provision of the time adjustment information, a message prompting the user to read out the display such as the systolic blood pressure may be displayed on the touch panel display 220 or output from the voice output unit 270. In addition, in a case where the time to be adjusted is short, for example, approximately one second, the display of blood pressure information (step S29) may be delayed slightly for the time adjustment processing.

### Answer Check Processing

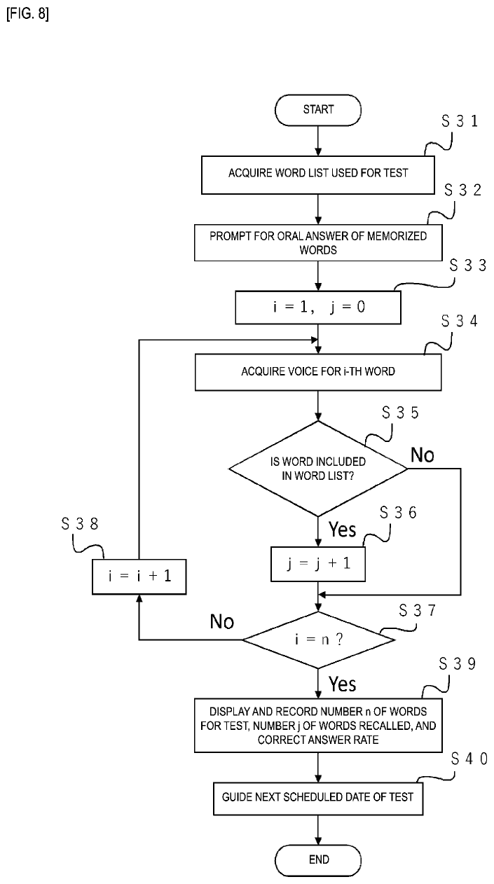

FIG. 8 is a flowchart illustrating a processing procedure for checking answers from the user to the provided cognitive function test information. The answer check processing described below, including other examples, is implemented by the control unit 240 of the smartphone 20 by executing a program stored in the storage unit 230. In other words, an answer check processing unit is configured by including the touch panel display 220, the storage unit 230, the clock unit 250, the voice input unit 260, and the voice output unit 270, and the control unit 240 cooperating with the units 220, 230, 250, 260, and 270.

In step S31, the word list used for the test is acquired from the cognitive function test result recording unit 232 of the storage unit 230.

Then, a message prompting the user to orally answer memorized words is displayed on the touch panel display 220 (step S32). For example, a message such as “Please speak memorized words” is displayed on the touch panel display 220. A message prompting the oral answer of the memorized words may be output via the voice output unit 270, and display and voice output may be combined.

Then, i=1 and j=0 are set (step S33).

Then, the voice of the user for the i-th word is acquired via the voice input unit 260 (step S34).

The acquired voice is analyzed to determine whether or not the word answered by the user is included in the word list acquired in step S31 (step S35). In step S35, in a case where the word is determined to be included in the word list, j=j+1 is set (step S36) and the processing proceeds to step S37. In step S35, in a case where the word is determined not to be included in the word list, then the processing proceeds to step S37.

In step S37, whether i=n is determined.

In step S37, in a case where i=n does not hold true, then i=i+1 is set (step S38) and the processing returns to step S34 to wait to acquire the voice for the i+1th word.

In step S37, in a case where i=n holds true, the number n of words used in the current cognitive function test, the number j of words successfully recalled by the user, and the correct answer rate j/n are displayed on the touch panel display 220, and are recorded in the cognitive function test result recording unit 232 of the storage unit 230 (step S39).

Then, the user is guided to the next scheduled date of the cognitive function test (step S40). For example, the touch panel display 220 displays a message such as “Thank you for taking the test. Well done. To obtain the final result, please run the test three more times. The next test will be performed on day X”. The message may be output via the voice output unit 270.

The next scheduled date of the test is recorded in the cognitive function test result recording unit 232 of the storage unit 230, and at the time of measuring the blood pressure information on the day, the next cognitive function tests are prompted. Thus, accuracy of evaluation is increased by conducting the cognitive function test a plurality of times.

### Test Result Determination Processing

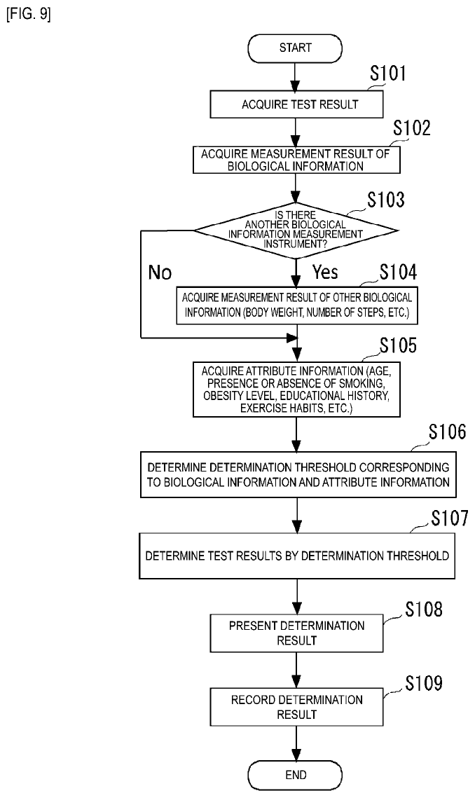

FIG. 9 is a flowchart illustrating an example of a processing procedure for determining the state of the cognitive function of the user from the result (for example, the correct answer rate j/n) of the cognitive function test. The determination processing described below, including other examples, is implemented by the control unit 240 of the smartphone 20 by executing a program stored in the storage unit 230. In other words, a cognitive function determination unit is configured by including the touch panel display 220, the storage unit 230, the clock unit 250, the voice input unit 260, and the voice output unit 270, and the control unit 240 cooperating with the units 220, 230, 250, 260, and 270.

In step S101, the correct answer rate is acquired from the cognitive function test result recording unit 232 of the storage unit 230. In step S102, the measured biological information (blood pressure information or the like) is acquired from the measurement result recording unit 231 of the storage unit 230. In step S103, the presence or absence of another biological information measurement instrument connected to the network is determined. For example, when there is biological information transferred from the other biological information measurement instrument with the smartphone 20 as a destination, the smartphone 20 determines that there is the other biological information measurement instrument. Otherwise, it is determined that there is no other biological information measurement instrument. In step S103, when it is determined that there is the other biological information measurement instrument (“Yes” in step S103), the process proceeds to step S104. Otherwise (“No” in step S103), the process proceeds to step S105.

In step S104, the measurement result of the biological information measured by the other biological information measurement instrument is acquired. For example, from the biological information measurement instrument 30, the measurement result 31 such as a body composition including a body weight and a body fat percentage, and a BMI value recorded in a memory or the like is acquired. In addition, the measurement result 41 of an exercise amount such as the number of steps or consumed calories recorded in a memory or the like is acquired from the biological information measurement instrument 40.

In step S105, the attribute information of the user is acquired from the attribute information storage unit 235 of the storage unit 230. The attribute information includes, for example, personal information such as age and gender of the user, and general information indicating daily “lifestyle” such as presence or absence of smoking, presence or absence of drinking, obesity level, educational history, and exercise habits. The presence or absence of smoking may include the number of cigarettes smoked per day or smoking history. The presence or absence of drinking may include the type of alcohol-containing beverage such as beer, sake, whisky, and the like, and the intake amount per day. The obesity level can be acquired from the biological information measurement instrument 30, but may be calculated based on the height, body weight, and the like input by the user. As the educational history, for example, a section to which type of last completed education belongs (middle school, high school, professional school, junior college, and university) can be exemplified. The educational history may include “business history”, “history related to occupation”, “information related to life history”, and the like. The exercise habits are typically the execution frequency of exercises, such as strolling, walking, swimming, and cycling, which are regularly performed on a weekly or monthly basis. However, the exercise habits may include information indicating walking distance and walking time among daily lifestyles. Note that the information related to the lifestyle may include, for example, the measurement frequency of the biological information or the frequency of the medication (the number of times taken actually to the number of times to be taken).

Such attribute information may be acquired periodically from other biological information measurement instruments connected to the network, or may be received via an input unit of the smartphone 20. In addition, at the time of measurement of biological information or at the time of the test of the cognitive function, it is also possible to acquire the attribute information through the voice output unit 270 or the voice input unit 260 during a dialogue with the user. For example, through interaction with the user, the presence or absence of regular meals, the presence or absence of regular sleep, the frequency of ingestion of fruits and vegetables, and the like can be acquired as the attribute information.

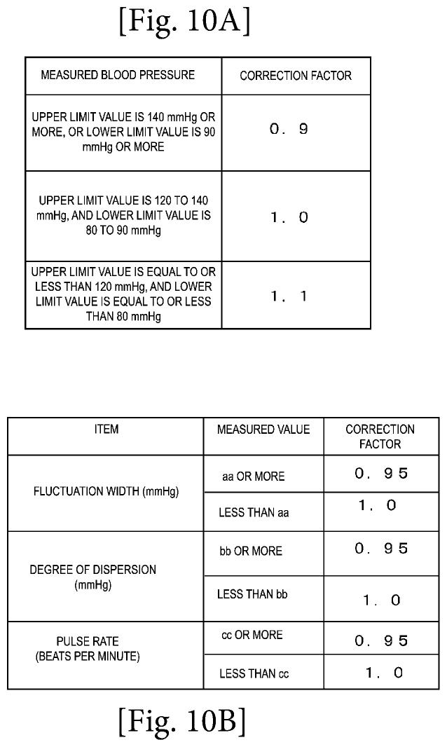

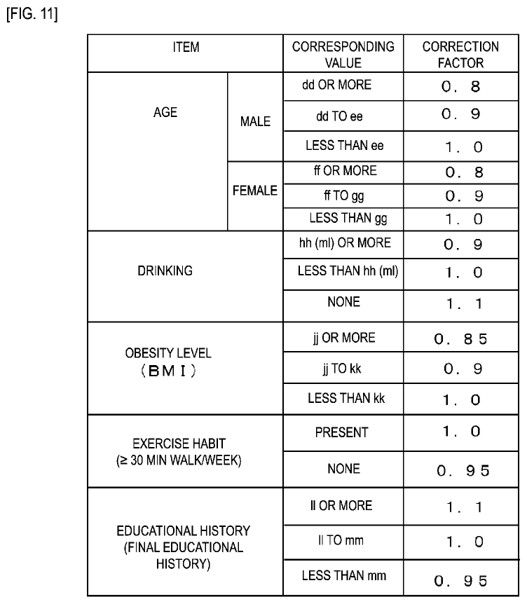

In step S106, a determination threshold for determining the states of the cognitive function corresponding to the biological information and the attribute information are determined. The determination threshold is determined, for example, using a correction factor stored in the determination correction table 236 of the storage unit 230. FIG. 10 and FIG. 11 illustrate an example of the determination correction table 236. FIG. 10(a) illustrates an example of a table of correction factors for the measurement result of the blood pressure value, and FIG. 10(b) illustrates an example of a table of correction factors related to the fluctuation width and the degree of dispersion of the measured blood pressure value and the pulse rate. Note that in the correction factor table in FIG. 10(b), “aa” to “cc” indicated in the item “measured value” are predetermined set values. FIG. 11 illustrates a table example of the correction factor for the attribute information. Also in FIG. 11, “dd” to “hh” and “jj” to “mm” indicated in “corresponding value” are predetermined set values. Here, the correction factor is, for example, a correction value of less than 1.0. However, the value used as the correction factor may be greater than 1.0. The correction factor has a value for each acquired biological information and type of attribute information.

Taking blood pressure information as an example, it is generally desirable that the upper limit value of the blood pressure value is about 120 mmHg of the systolic blood pressure and the lower limit value thereof is about 80 mmHg of the diastolic blood pressure. The blood pressure value diagnosed as hypertension is such that the upper limit value of systolic blood pressure is 140 mmHg or more, or the lower limit value of diastolic blood pressure is 90 mmHg or more. Therefore, as illustrated in FIG. 10(a), the correction factor for the blood pressure information is differentiated such that the correction factor is “0.9” when the blood pressure value is diagnosed as hypertension, the correction factor is “1.1” when the blood pressure value is desirable, and the correction factor is “1.0” when the blood pressure value is between hypertension and the desirable blood pressure value. The same applies to the fluctuation width, which is the difference between the systolic blood pressure value and the diastolic blood pressure value of the blood pressure values, the fluctuation of the measured systolic blood pressure value or diastolic blood pressure value (for example, an inter-day fluctuation or an intra-day fluctuation of the systolic blood pressure value or the diastolic blood pressure value), and the pulse rate. For example, the threshold may be corrected using these values instead of or in combination with the blood pressure value.

Note that such a correction factor can be determined experimentally. When the blood pressure value is such that the burden on the blood vessel wall through which blood flows is high, the value of the correction factor can be made relatively low.

The same applies to the attribute information illustrated in FIG. 11. For example, the relative value of the correction factor changes according to an age group to which the age belongs. For example, the higher the age, the relatively lower the value of the correction factor. For gender, for example, the same value can be set up to a certain age, and a gender difference can be set after a certain age.

For each of the presence or absence of smoking, the presence or absence of drinking, the obesity level, and the exercise habit, the higher the risk factor for the lifestyle-related disease is, the lower the value of the correction factor is relatively. For example, regarding a smoking habit, when the subject is presently a smoker, the value of the correction factor is relatively the low. In addition, the correction factor for smoking history is relatively higher than the correction factor for the current smoking habits and relatively lower than the correction factor for the case where there is no smoking habit. As for the presence or absence of drinking, the value of the correction factor becomes relatively low when the intake amount per day exceeds an appropriate amount. For example, the obesity level becomes relatively low according to each stage of the obesity level divided by the BMI value. In the exercise habit, for example, the correction factor when there is no exercise habit is relatively lower than that when there is an exercise habit. In the educational history, for example, in a case where the category to which the final school education course belongs is a professional school or the like, the correction factor is relatively higher than in a case where the category to which the most recently completed school education belongs is not a vocational school or the like.

In step S106, the determination threshold may be determined by obtaining the correction factor only for the biological information, or the determination threshold may be determined from the correction factor obtained by combining a plurality of pieces of biological information. Furthermore, when combining the biological information and the attribute information, a single piece of attribute information may be combined, or a plurality of pieces of attribute information of different types may be combined.

In addition, in the determination of the determination threshold, instead of the correction factor, the determination threshold may be determined based on a point value assigned in advance for each piece of biological information or attribute information. For example, a relative index indicating a difference in degree can be given as a point value, such as “+10” when there is no smoking habit, “−10” when there is a smoking habit, and “−5” when there is a history of smoking. Then, a relatively increased or decreased determination threshold may be determined based on the total point value added in accordance with the biological information or the attribute information. As the determination threshold, a plurality of thresholds divided in stages can be set.

In step S107, the test result of the cognitive function is determined using the determined determination threshold. For example, when the correct answer rate is equal to or greater than the determination threshold, it is determined that there is no problem in the state of the cognitive function. Otherwise, it is determined that the state of the current cognitive function has a problem.

In addition, when the determination threshold is set to a plurality of stages in the order of A, B, and C, for example, it is determined that there is no problem in the state of the cognitive function when the correct answer rate is equal to or greater than the determination threshold A. When the correct answer rate is less than the determination threshold C, for example, it is determined that there is a problem in the current state of the cognitive function. A correct answer rate that is equal to or greater than the determination threshold C and less than the determination threshold A may be determined based on, for example, a transition of the correct answer rate in a certain period.

However, the determination of the test result of the cognitive function can be determined in an aspect other than the determination threshold. For example, in the answer check processing, the state of the cognitive function of the user may be determined based on the presence or absence of memory for a predetermined word or illustration. Such words and illustrations related to the determination of the cognitive function can be set in advance. By determining the state of the cognitive function by using the presence or absence of memory for a predetermined word or illustration, it becomes possible to perform determination based on memory between words or illustrations having relevance, memory between words or illustrations not having relevance, or the like, and improvement in determination accuracy can be expected.

In step S108, the state of the cognitive function determined by the determination threshold is presented. For example, the determination result can be displayed on the touch panel display 220 together with the biological information, the attribute information, and the test result (correct answer rate) related to the determination. In addition, a notification such as “there is no problem with your cognitive function” or “there is a problem with your cognitive function. Please get tested at a medical institution” may be delivered through the voice output unit 270. When the correct answer rate is equal to or higher than the determination threshold C and lower than the determination threshold A, a notification such as “While there isn't necessarily a problem you're your cognitive functioning, please reevaluate your lifestyle habits” may be delivered. In addition, a notification may be delivered to provide the user with self-awareness to re-evaluate their lifestyle habits, such as a specific approach (for example, “Let's walk for about 15 minutes every day”) that is considered conducive to prevention of deterioration of the cognitive function.

In step S109, the determination result is recorded. For example, in the cognitive function test result recording unit 232 of the storage unit 230, the determination threshold and the determination result used for the determination are recorded in association with the cognitive function test result.

As described above, according to the present embodiment, it is possible to determine the state of the cognitive function of the user with respect to the result of the cognitive function test using the determination threshold corrected in accordance with the blood pressure information or the attribute information acquired as the biological information. It is possible to determine the cognitive function in consideration of an element that is included in biological information measured in daily healthcare management and attribute information related to physical characteristics and lifestyle habits of the user, and that is considered to be changed prior to or accompanied by the onset of dementia or a change in the state of the cognitive function. In the present embodiment, early detection of dementia can be expected because the cognitive function test can be performed using the opportunity of measurement of the blood pressure information performed as a part of routine healthcare management.

### Second Embodiment

A second embodiment of the present invention will be described below. The same reference signs are used for configurations that are the same as those of the first embodiment, and detailed descriptions thereof are omitted.

The present embodiment is the same as the first embodiment in the configuration of the biological information measurement system, the overall flow from the provision of the cognitive function test information through the measurement of the blood pressure information to the check of answers to the test information and the determination of the test result, and the contents of the blood pressure measurement processing. The present embodiment differs from the first embodiment in the contents of the cognitive function test, and thus processing for cognitive function tests and check processing for answers to the test information will be described.

### Cognitive Function Test Information Provision Processing

The cognitive function test of the present embodiment is referred to as word recognition, in which the user is prompted to memorize a plurality of words displayed, and after a predetermined amount of time or after a predetermined interference task, a plurality of words are displayed that include the previously displayed words and dummy words, and the user is prompted to answer whether the currently displayed words are the previously displayed words.

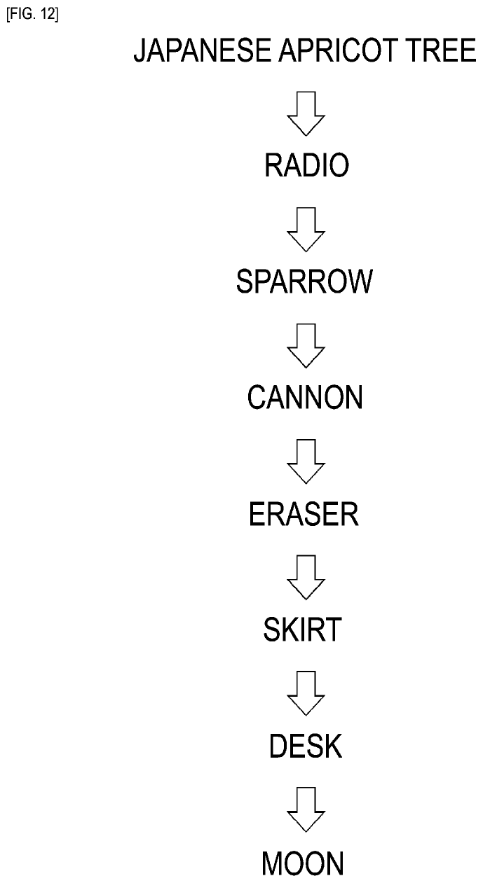

The provision processing for the cognitive function test information in the present embodiment is similar to that in FIG. 3. In this case, for example, the number n of words displayed is eight, and eight words including, “Japanese including “apricot tree”, “radio”, “sparrow”, “cannon”, “eraser”, “skirt”, “desk”, and “moon” are sequentially displayed on the touch panel display 220 of the smartphone 20 in this order, as illustrated in FIG. 12. These words are extracted from at least three or more categories.

### Answer Check Processing

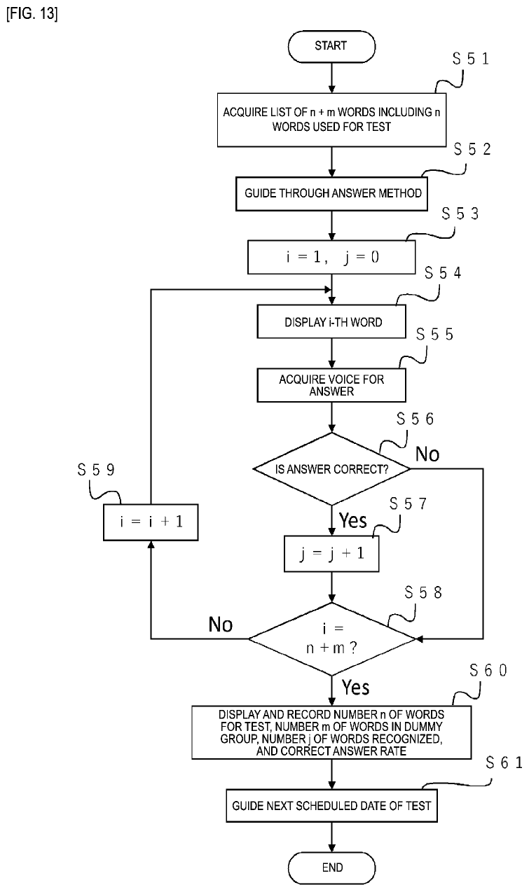

The blood pressure measurement processing is similar to that in the first embodiment, and thus the description of the blood pressure measurement processing is omitted, and the answer check processing will be described below with reference to FIG. 13.

First, n words (correct answer group) used to provide the cognitive function test information are read from the cognitive function test result recording unit 232 of the memory unit 230, and from the group of words stored in the cognitive function test information storage unit 233, m words (dummy group) not included in the correct answer group are extracted, and a word list including n+m words is acquired (step S51).

Then, a message is displayed on the touch panel display 220, the message guiding the user through the answer method such that in a case where the word displayed on the touch panel display 220 is the word displayed before the blood pressure measurement, the user verbally answers “yes”, whereas in a case where the word displayed on the touch panel display 220 is not the word displayed before the blood pressure measurement, the user verbally answers “no” (step S52). The message guiding the user through the answer method may be output via the voice output unit 270, and the display and the voice output may be combined.

Then, i=1 and j=0 are set (step S53).

Then, the i-th word is displayed on the touch panel display 220 of the smartphone 20 (step S54). In this case, as described below, any one of words included in the correct answer group or words not included in the correct answer group is displayed.

Then, the voice of the user indicating the answer “yes” or “no” is acquired via the voice input unit 260 (step S55).

The voice acquired is analyzed to determine whether the answer is correct (step S56). In other words, in step S54, in a case where the word displayed on the touch panel display 220 is included in the correct answer group and the answer acquired is “yes”, the answer is determined to be correct, and in a case where the answer acquired is “no”, the answer is determined to be incorrect. Further, in step S54, in a case where the word displayed on the touch panel display 220 is included in the dummy group and the answer acquired is “no”, the answer is determined to be correct, and in a case where the answer acquired is “yes”, the answer is determined to be incorrect.

In step S56, in a case where the answer is determined to be correct, j=j+1 is set (step S57), and the processing proceeds to step S58.

In step S56, in a case where the answer is determined to be incorrect, then the processing proceeds to step S58.

In step S58, whether i=n+m is determined.

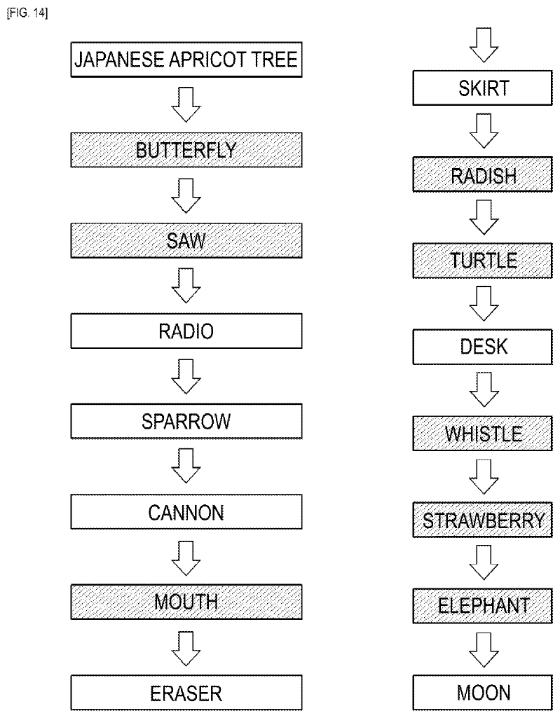

In step S58, in a case where i=n+m does not hold true, then i=i+1 is set (step S59) and the processing returns to step S54 to display the i+1th word on the touch panel display 220. In this manner, e.g., as illustrated in FIG. 14, 16 words including, “Japanese apricot tree”, “butterfly”, “saw”, “radio”, “sparrow”, “cannon”, “mouth”, “eraser”, “skirt”, “radish”, “turtle”, “desk”, “whistle”, “strawberry”, “elephant”, and “moon” are displayed sequentially on the touch panel display 220 in this order. In this case, m is eight, and eight shaded words are extracted as a dummy group.

In step S58, in a case where i=n+m holds true, the number n of words used in the current cognitive function test, the number m of words added as dummies, the number j of words that can be recognized by the user, and the correct answer rate j/(n+m) are displayed on the touch panel display 220, and are recorded in the cognitive function test result recording unit 232 of the storage unit 230 (step S60).

Then, the user is guided to the next scheduled date of the cognitive function test (step S61). For example, the touch panel display 220 displays a message such as “Thank you for taking the test. Well done. To obtain the final result, please run the test three more times. The next test will be performed on day X”. The message may be output via the voice output unit 270.

The next scheduled date of the test is recorded in the cognitive function test result recording unit 232 of the storage unit 230, and at the time of measuring the blood pressure information on the day, the next cognitive function tests are prompted.

In the present embodiment as well, the number n of words displayed in the provision processing for the cognitive function test information is not limited to eight, and may be set as appropriate. In a case where the similar tests are repeated a plurality of times, the number of displayed words may be varied each time, for example, eight words on the first test, six words on the second test, and 12 words on the third test. Additionally, the number of words extracted as a dummy group may be the same number as the number of words displayed in the provision processing for the cognitive function test information, but the number of dummies can be increased or decreased to adjust the difficulty level.

In this manner, even in the present embodiment, it is possible to determine the cognitive function in consideration of an element that is included in biological information measured in daily healthcare management and attribute information related to physical characteristics and lifestyle habits of the user, and that is considered to be changed with the onset of dementia or a change in the state of the cognitive function.

### Third Embodiment

A third embodiment of the present invention will be described below. The same reference signs are used for configurations that are the same as those of the first embodiment, and detailed descriptions thereof are omitted.

The present embodiment is the same as the first embodiment in the configuration of the biological information measurement system, the overall flow from the provision of the cognitive function test information through the measurement of the blood pressure information to the check of answers to the test information, and the contents of the blood pressure measurement processing. The present embodiment differs from the first embodiment in the contents of the cognitive function test, and thus processing for cognitive function tests and check processing for answers to the test information will be described.

### Cognitive Function Test Information Provision Processing

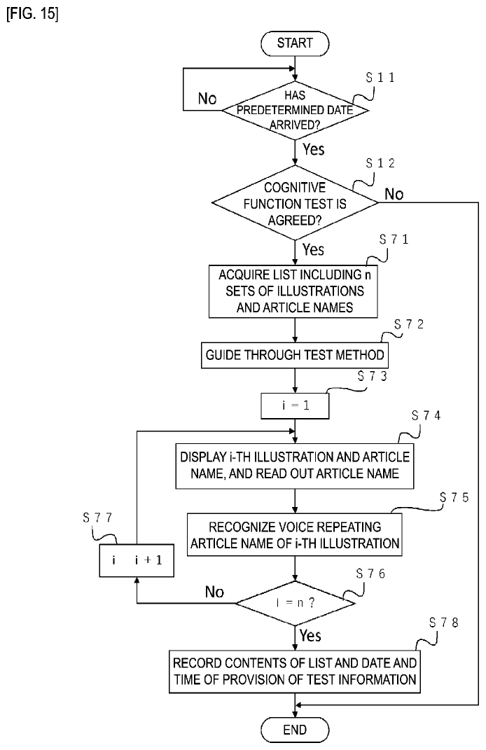

FIG. 15 is a flowchart illustrating a processing procedure for provision of the cognitive function test information. The cognitive function test of the present embodiment is referred to as word recalling using an image, in which the user is prompted to memorize the article name of displayed illustrations and to verbally speak the previously displayed illustration after a predetermined amount of time or predetermined interference task.

The processing in step S11 and step S12 is similar to that in the first embodiment, and thus the descriptions thereof are omitted.

Then, a list of n illustrations and the article names of the illustrations stored in the cognitive function test information storage unit 233 of the storage unit 230 is acquired (step S71). The cognitive function test information storage unit 233 may store the list, or may extract n sets of illustrations and article names from a group including a plurality of sets of illustrations and article names and form the sets into the list.

Then, the cognitive function test method to be performed is displayed on the touch panel display 220 of the smartphone 20 to guide through the method (step S72). Here, a message for guiding a method of the cognitive function test such as “From now, n illustrations will be displayed one by one. The article name of the displayed illustration is read out, so please repeat and memorize the article name” is displayed. The message may also be output by the voice output unit 270 of the smartphone 20.

Then, i=1 is set (step S73).

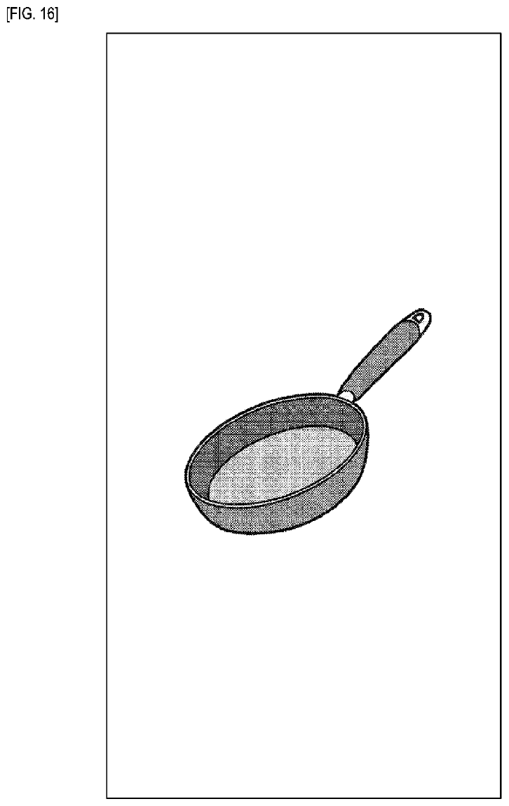

Then, as illustrated in FIG. 16, the first illustration of the list acquired in step S71 is displayed on the touch panel display 220, and the voice of reading out the article name of the illustration is output from the voice output unit 270 (step S74). In this case, an illustration of a frying pan is displayed on the touch panel display 220, and the voice “frying pan”, corresponding to an article name, is output from the voice output unit 270. At this time, a message may similarly be displayed that prompts the user to repeat the article name.

Then, from the voice acquired via the voice input unit 260 of the smartphone 20, the voice of the user repeating the article name of the i-th illustration is recognized (step S75). In a case where the voice of the user reading out the i-th illustration fails to be recognized within a predetermined amount of time, then a message prompting the user to read out the displayed illustration may be displayed on the touch panel display 220 or output via the voice output unit 270. Additionally, in a case where the voice of the user reading out the i-th illustration fails to be recognized within the predetermined amount of time, then the processing may proceed to step S76.

Once the voice of the user repeating the article name of the i-th illustration is recognized, then whether i=n is determined (step S76).

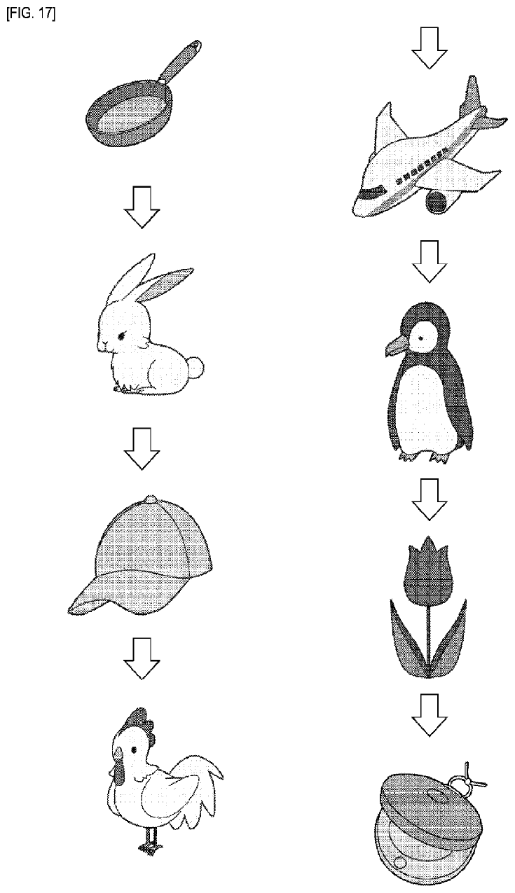

In step 76, if i=n is not satisfied, i=i+1 is set (step S77), the process returns to step S74, and the processing after step S74 are repeated for the next illustration and article name in the list acquired in step S71. In this manner, as illustrated in FIG. 17, different illustrations are displayed sequentially on the touch panel display 220 of the smartphone 20. In this case, the list includes eight illustrations of a frying pan, a rabbit, a cap, a chicken, an airplane, a penguin, a tulip, and a castanet. The illustrations displayed at this time all belong to different categories. The number n of illustrations displayed is not limited to eight, and may be set as appropriate. In a case where similar tests are repeated a plurality of times, the number of displayed illustrations may be increased each time, for example, eight illustrations on the first test and the 12 illustrations on the second test.

If i=n in step S76, the contents of the list acquired in step S71 and the date and time when the test information was provided are stored in the cognitive function test result recording unit 232 of the storage unit 230 (step S78). In this case, the date and time of provision of the test information is, for example, the date and time of recognition of the voice of reading out the nth illustration. Then, the provision processing for the cognitive function test information is ended.

### Answer Check Processing

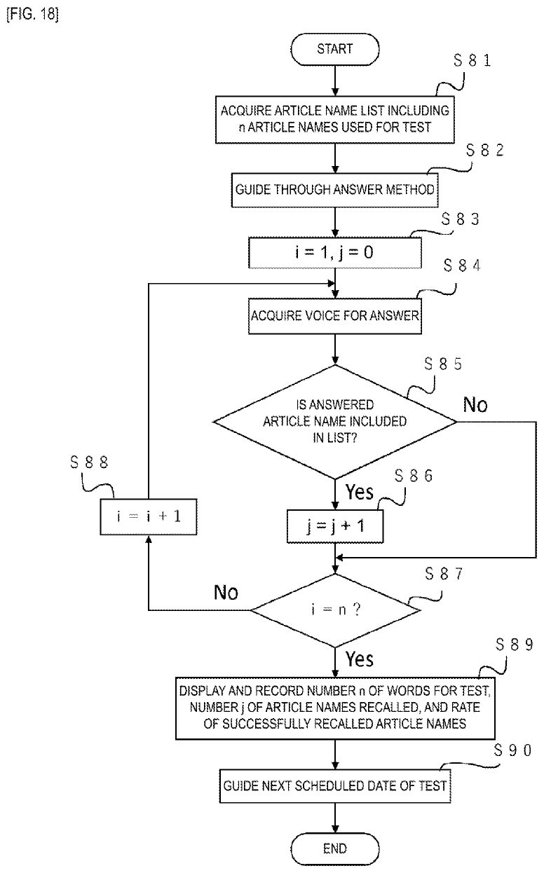

The blood pressure measurement processing is similar to that in the first embodiment, and thus the description of the blood pressure measurement processing is omitted, and the answer check processing will be described below with reference to FIG. 18.

First, an article name list including n article names used to provide the cognitive function test information is read and acquired from the cognitive function test result recording unit 232 of the storage unit 230 (step S81).

Then, a message prompting the user to orally answer memorized article names is displayed on the touch panel display 220 (step S82). For example, a message such as “Please say the names of the articles that you can recall” is displayed on the touch panel display 220. The message prompting the user to orally answer the memorized article names may be output via the voice output unit 270, and the display and the voice output may be combined.

Then, i=1 and j=0 are set (step S83).

Then, the voice of the user for the i-th illustration is acquired via the voice input unit 260 (step S84).

The acquired voice is analyzed to determine whether or not the article name indicated by the voice is included in the article name list acquired in step S81 (step S85).

In step S85, in a case where the article name is determined to be included in the article name list, j=j+1 is set (step S86) and the processing proceeds to step S87.

In step S85, in a case where the article name is determined not to be included in the article name list, then the processing proceeds to step S87.

In step S87, whether i=n is determined.

In step S87, in a case where i=n does not hold true, then i=i+1 is set (step S88) and the processing returns to step S84 to wait to acquire the voice for the i+1th illustration.

In step S87, in a case where i=n holds true, the number n of article names used in the current cognitive function test, the number j of article names successfully recalled by the user, and the correct answer rate j/n are displayed on the touch panel display 220, and are recorded in the cognitive function test result recording unit 232 of the storage unit 230 (step S89).

Then, the user is guided to the next scheduled date of the cognitive function test (step S90). For example, the touch panel display 220 displays a message such as “Thank you for taking the test. Well done. To obtain the final result, please run the test three more times. The next test will be performed on day X”. The message may be output via the voice output unit 270.

The next scheduled date of the test is recorded in the cognitive function test result recording unit 232 of the storage unit 230, and at the time of measuring the blood pressure information on the day, the next cognitive function tests are prompted. Thus, accuracy of evaluation is increased by conducting the cognitive function test a plurality of times.

In this manner, even in the present embodiment, it is possible to determine the cognitive function in consideration of an element that is included in biological information measured in daily healthcare management and attribute information related to physical characteristics and a lifestyle of the user, and that is considered to be changed with the onset of dementia or a change in the state of the cognitive function.

### Fourth Embodiment

In the present embodiment, the biological information has been acquired by receiving transfer of biological information already measured by the other biological information measurement instrument. In other words, the biological information acquisition processing is implemented as a transferred biological information reception processing. The same reference signs are used for configurations and processing that are common as those of the first embodiment, and the detailed descriptions thereof are omitted. The biological information includes, for example, but is not limited to, body weight, body composition, exercise amount, or body temperature.

The schematic configuration of the biological information measurement system 1 used as a biological information acquisition device is same as that in the first embodiment illustrated in FIG. 1. However, in the present embodiment, the blood pressure monitor 10 receives the measurement result as the biological information already measured by the other cooperating biological information measurement instrument via the communication unit 130 and stores the measurement result in the measurement result recording unit 170 of the storage unit 160. Then, the blood pressure monitor 10 transfers the measurement result stored in the measurement result recording unit 170 to the smartphone 20, and the smartphone 20 acquires the biological information measured by the other biological information measurement instrument.

The device storing the biological information measured by the other biological information measurement instrument may be a device having a biological information measurement function, but may be a device not having a biological information measurement function.

The processing procedure for the biological information acquisition method including the cognitive function test processing, the biological information acquisition processing, and the answer check processing is similar to that in the first embodiment illustrated in FIG. 2.

### Cognitive Function Test Information Provision Processing

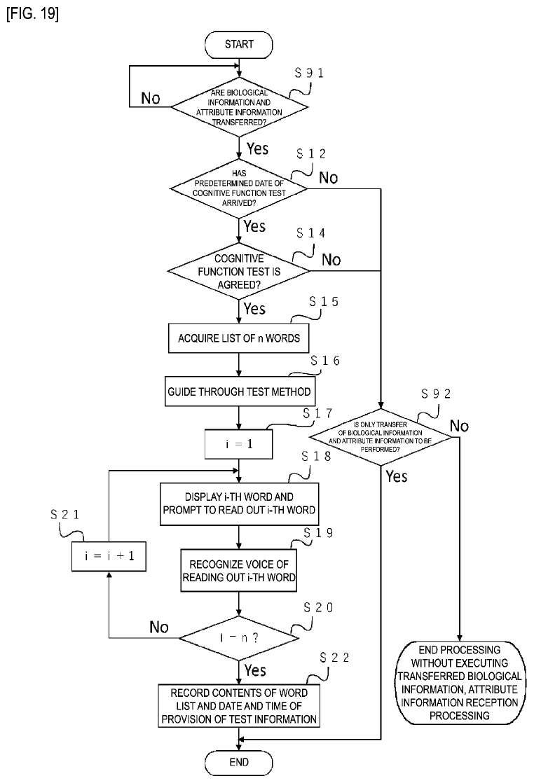

FIG. 19 illustrates the cognitive function test information provision processing.

Only differences from the cognitive function test information provision processing of the first embodiment illustrated in FIG. 3 will be described. In the present embodiment, a message “Would you like to receive biological information and attribute information?” is displayed on the touch panel display 220 of the smartphone 20, or the voice for the message is output from the voice output unit 270. Then, the user is asked to agree with the transfer of the biological information and the attribute information (step S91).

The processing in step S12 is similar to that in the first embodiment. In step S12, in a case where it is determined that the cognitive function test date is determined not to have arrived, the user is prompted by the touch panel display 220 or the voice output unit 270 to check whether only transfer of the biological information is to be performed (step S92).

In step S92, in a case where an input indicating that only the transfer of the biological information and the attribute information is to be performed is received from the user via the touch panel display 220 or via the voice input unit 260, the cognitive function test information provision processing is ended. Then, transferred biological information/attribute information reception processing to be described later is performed. In this case, in a case where the transferred biological information/attribute information reception processing ends, the processing is ended without performing the answer check processing. In step S92, in a case where an input indicating that only the transfer of the biological information/attribute information is not to be performed is received from the user via the touch panel display 220 or via the voice input unit 260, the processing is ended without performing the reception processing of the transferred biological information/attribute information.

### Transferred Biological Information/Attribute Information Reception Processing

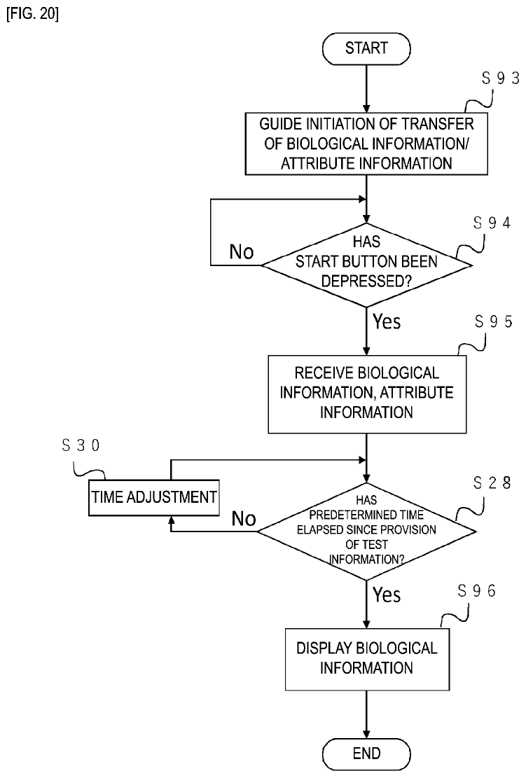

FIG. 20 is a flowchart illustrating a processing procedure for reception of the transferred biological information/attribute information. The transferred biological information/attribute information reception processing described below is implemented by the control unit 240 of the smartphone 20 executing a program stored in the storage unit 230 and cooperating with the blood pressure monitor 10. At this time, in the blood pressure monitor 10, the control unit 150 similarly executes a program stored in the storage unit 160. A biological information acquisition unit includes the touch panel display 220, the storage unit 230, the clock unit 250, the voice input unit 260 and the voice output unit 270, the communication unit 210, and the control 240 that are belonging to the smartphone 20, in which the control unit 240 cooperates with the units 220, 230, 250, 260, 270, and 210. The biological information acquisition unit further includes at least the storage unit 160, the communication unit 130, and the control unit 150 that are belonging to the blood pressure monitor 10, in which the control unit 150 cooperates with the units 160 and 130.

First, the initiation of the transfer of the biological information/attribute information is guided to the user (step S93). At this time, the “START” button is displayed on the touch panel display 220 of the smartphone 20, and a message “Press the START button or say ‘start’ to initiate transfer.”, which guides the initiation of the transfer, is displayed on the touch panel display 220 or output from the voice output unit 270.

The smartphone 20 waits for the user to depress the “START” button displayed on the touch panel display 220 of the smartphone 20 or waits for the user to input the voice “start” via the voice input unit 260 (step S94). When depression of the “START” button or the voice “start” is detected, the biological information and the attribute information already measured by the other biological information measurement instrument are received from the measurement result recording unit 170 of the blood pressure monitor 10 (step S95).

The processing in steps S28 and S30 is similar to that in the first embodiment illustrated in FIG. 6.

In step S28, in a case where the determination is made that a predetermined amount of time has elapsed since the date and time of provision of the cognitive function test information, the biological information received in step S95 is displayed on the touch panel display 220 of the smartphone 20 (step S96), and the processing is ended. In step S96, the received biological information may be output via the voice output unit 270.

As described above, according to the present embodiment, the cognitive function test can be performed by using the opportunity to transfer the biological information and the attribute information measured as a part of routine healthcare management. Then, it is possible to determine the cognitive function in consideration of an element that is included in the measured biological information and the attribute information related to physical characteristics and a lifestyle of the user, and that is considered to be changed with the onset of dementia or a change in the state of the cognitive function.

### MODIFIED EXAMPLE

The processing in step S28 and step S30 in the blood pressure information measurement processing may be performed in another procedure in the blood pressure information measurement processing.

The contents of the cognitive function test are not limited to the contents described in the first embodiment, the second embodiment, and the third embodiment, and any other cognitive function test may be employed, and the cognitive function tests described in the first embodiment, the second embodiment, and the third embodiment may be combined.

Additionally, by changing test information such as the numbers of words and illustrations provided as the cognitive function test information, the difficulty level, and the like, the amount of time required after the end of the cognitive function test information provision processing until the initiation of the answer check processing can be extended or shortened. Thus, the test information may be changed to match the time of the blood pressure information measurement processing.

In the first embodiment, the biological information measurement system that has been described includes, as the biological information measurement instrument, the blood pressure monitor that measures the blood pressure information. The present invention can also be applied to a biological information measurement system that includes, as the biological information measurement instrument, a body weight meter measuring the body weight as the biological measurement information, a body composition analyzer measuring the body composition such as the body fat percentage, an exercise meter measuring the exercise amount such as the number of steps or calorie consumption, and a clinical thermometer measuring the body temperature.

In addition, in the first embodiment, the blood pressure monitor 10 is configured to measure blood pressure information alone, but in FIG. 1, the blood pressure monitor 10 can be configured such that the input unit 140 and the display unit 120 are omitted and input of operation information or the like and the display of various information are performed on the smartphone 20 side. Additionally, in FIG. 1, the blood pressure monitor 10 can be configured such that the measurement result recording unit 170 is omitted and the measurement results are recorded on the smartphone 20 side.

The biological information measurement system according to the first embodiment includes a smartphone 20 as a terminal, but the terminal is not limited thereto, and may be a mobile information terminal such as a tablet terminal or a laptop computer, or a stationary information terminal.

In addition, as illustrated in FIG. 2, the blood pressure information is measured between the provision of the cognitive function test information and the checking of answers from the user against the cognitive function test information. In a case where each of the processing are performed in this order, the provision of the cognitive function test information and the checking of the first answer from the user against the cognitive function test information may be performed before the measurement of the blood pressure information, and the checking of the second answer from the user to the cognitive function test information may be performed after the measurement of the blood pressure information. In a case where cognitive function tests are conducted that vary in the time required to answer a question is and interference tasks, for a test that takes longer to complete, the first answer checking is performed before the measurement of the blood pressure information, and for a test that takes longer to complete, the second answer checking may be performed after the measurement of the blood pressure information. In addition, for the same cognitive function test that varies in the time required to give an answer and varies in interference tasks, the answer check may be performed before and after the measurement of the blood pressure information.

Additionally, in the blood pressure information measurement processing illustrated in FIG. 6, the initiation of the blood pressure measurement is guided after the end of the cognitive function test information provision processing. In the biological information measurement processing including, as an example, the blood pressure information measurement processing as described above, an exercise task such as stationary stepping or curb stepping exercises may be assigned to the user before the biological information is measured, and after the task, the biological information may be measured. This allows the exercise task to be incorporated into the measurement of the biological information as a method for time adjustment. In this case, time adjustment can be made by displaying the timing of stepping and the number of steps in the step-ups exercise on the touch panel display 220 of the smartphone 20 or outputting the timing of stepping and the number of steps from the voice output unit 270.

### REFERENCE SIGNS LIST

1 Biological information measurement system
10 Blood pressure monitor
20 Smartphone
110 Sensor unit
130 Communication unit
150 Control unit
160 Storage unit
210 Communication unit
220 Touch panel display
230 Storage unit
240 Control unit
250 Clock unit
260 Voice input unit
270 Voice output unit

1. A biological information acquisition device configured to perform a cognitive function test, the biological information acquisition device comprising:
a processor configured to:
test a cognitive function of a user;
perform biological information acquisition processing to acquire biological information of the user; and
determine a state of the cognitive function, wherein
the processor determines the state of the cognitive function of the user by using a result of the cognitive function test and a determination threshold for determining the cognitive function and corrects the determination threshold based on the biological information acquired from the user.

2. The biological information acquisition device according to claim 1,
wherein the processor is further configured to acquire attribute information related to an attribute of the user, and wherein
the processor further determines the state of the cognitive function of the user based on the attribute information related to the attribute of the user.

3. The biological information acquisition device according to claim 1, wherein the processor is further configured to:
acquire a plurality of types of biological information related to the user, and
determine the state of the cognitive function of the user based on the plurality of types of biological information that is acquired.

4. The biological information acquisition device according to claim 2, wherein the processor is further configured to:
acquire at least one or more pieces of biological information measured by the other biological information measurement instrument that is communicably linked, and
acquire the attribute information related to the attribute of the user via the other biological information measurement instrument that is communicably linked.

5. The biological information acquisition device according to claim 1, wherein
processor is further configured to correct the determination threshold based on attribute information related to an attribute of the user.

6. The biological information acquisition device according to claim 1, wherein
the processor is further configured to correct the determination threshold based on the plurality of types of biological information that is acquired.

7. A biological information acquisition method capable of performing a cognitive function test, the biological information acquisition method comprising:
testing a cognitive function of a user;
acquiring biological information of the user; and
determining a state of the cognitive function of the user by using a result of the cognitive function test and a determination threshold for determining the cognitive function, and correcting the determination threshold based on the biological information acquired from the user.

8. The biological information acquisition device according to claim 2, wherein the processor is further configured to:
acquire a plurality of types of biological information related to the user, and
determine the state of the cognitive function of the user based on the plurality of types of biological information that is acquired.

9. The biological information acquisition device according to claim 2, wherein
processor is further configured to correct the determination threshold based on the attribute information related to the attribute of the user.

10. The biological information acquisition device according to claim 3, wherein
the processor is further configured to correct the determination threshold based on attribute information related to an attribute of the user.

11. The biological information acquisition device according to claim 4, wherein
the processor is further configured to correct the determination threshold based on the attribute information related to the attribute of the user.

12. The biological information acquisition device according to claim 8, wherein
the processor is further configured to correct the determination threshold based on the attribute information related to the attribute of the user.

13. The biological information acquisition device according to claim 2, wherein
the processor is further configured to correct the determination threshold based on the plurality of types of biological information that is acquired.

14. The biological information acquisition device according to claim 3, wherein
the processor is further configured to correct the determination threshold based on the plurality of types of biological information that is acquired.

15. The biological information acquisition device according to claim 4, wherein
the processor is further configured to correct the determination threshold based on the plurality of types of biological information that is acquired.

16. The biological information acquisition device according to claim 5, wherein
the processor is further configured to correct the determination threshold based on the plurality of types of biological information that is acquired.

17. The biological information acquisition device according to claim 8, wherein
the processor is further configured to correct the determination threshold based on the plurality of types of biological information that is acquired.

18. The biological information acquisition device according to claim 9, wherein
the processor is further configured to correct the determination threshold based on the plurality of types of biological information that is acquired.

19. The biological information acquisition device according to claim 10, wherein
the processor is further configured to correct the determination threshold based on the plurality of types of biological information that is acquired.

20. The biological information acquisition device according to claim 11, wherein
the processor is further configured to correct the determination threshold based on the plurality of types of biological information that is acquired.
